(19) United States
(12) Patent Application Publication
(10) Pub. No.: US 2016/0022554 A1
(43) Pub. Date: Jan. 28, 2016

(54) AGENT FOR THE TEMPORARY SHAPING OF KERATIN-CONTAINING FIBRES

(71) Applicant: Henkel AG & Co., KGaA, Duesseldorf (DE)

(72) Inventors: Thorsten Knappe, Schenefeld (DE); Julia Bibiane Lange, Sievershuetten (DE)

(21) Appl. No.: 14/851,044

(22) Filed: Sep. 11, 2015

Related U.S. Application Data
(63) Continuation of application No. PCT/EP2014/052045, filed on Feb. 3, 2014.

(30) Foreign Application Priority Data
Mar. 19, 2013 (DE) 10 2013 204 806.8

(51) Int. Cl. A61K 8/39; A61K 8/06; A61K 8/34; A61Q 5/06; A61K 8/92

(52) U.S. Cl. CPC A61K 8/39; A61Q 5/06; A61K 8/92; A61K 8/345; A61K 8/068

(57) ABSTRACT

The invention relates to a cosmetic composition for the temporary shaping of keratin-containing fibers, including (a) at least one emulsifier (a) from the group of the ethoxylated unsaturated C16-C18 fatty alcohols with an average degree of ethoxylation between 4 and 6; (b) at least one emulsifier (b) from the group of ethoxylated saturated C16-C18 fatty alcohols with an average degree of ethoxylation between 12 and 30, characterized in that the weight ratio of emulsifier (a) to emulsifier (b) ranges from 2:1 to 1:5, preferably from 1:1 to 1:2, and particularly suitable for the temporary shaping of keratin-containing fibers, particularly human hair.

### FIELD OF THE INVENTION

The present invention generally relates to cosmetic compositions for temporary shaping of keratinic fibers, in particular human hair.

### BACKGROUND OF THE INVENTION

Styling agents for shaping keratin-containing fibers have long been known and are used in a variety of embodiments for creating, freshening and fixation of hair styles, which can only be obtained with many types of hair by using hair-setting active ingredients. Hair treatment agents play an important role, both those which shaping the hair permanently and those that shape the hair temporarily.

The agents for temporary shaping are available in various forms, wherein hair waxes and hair gels are popular, in particular the propellant gas-free agents. From the consumer's standpoint, these agents differ with regard to their product optics and product haptics. The optics and haptics of a hair cosmetic, in addition to other sensory properties such as odor, are an important means for supporting product and brand name promise and thus play an important role in the commercial success of the product. For this reason, the optics and haptics of the styling agent are not usually the random result of a combination of specific active ingredients but instead are the result of a targeted product development.

Emulsifiers are often added to obtain cosmetic agents having the desired sensory properties. This is true in particular in the case of gelatinous, in particular transparent gelatinous hair styling agents. In this context, emulsifiers having phosphate groups such as oleth-3 phosphate, for example, have gained widespread popularity. However, the use of these emulsifiers having phosphate groups can lead to skin irritation.

The object of this patent application was therefore to make available cosmetic agents for temporary shaping of keratinic fibers with reduced potential for irritation; these agents are to be characterized by extraordinary optics and haptics as well as by good cosmetic properties.

It has been found that these objects can be achieved by a combination of two specific addition products of polyethylene oxide onto fatty alcohol if a certain weight ratio of these fatty alcohol ethoxylates is maintained.

Hair styling agents which contain combinations of specific fatty alcohol ethoxylates in addition to other ingredients are described, for example, in the Unexamined German Patent DE 10 2004 004 394 A1 (Wella) and the European Patent Application EP 1 800 648 A1 (Beiersdorf).

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Cosmetic composition for temporary shaping of keratin-containing fibers, including at least one emulsifier a) from the group of ethoxylated unsaturated C16-18 fatty alcohols with an average degree of ethoxylation of 4 to 6; at least one emulsifier b) from the group of ethoxylated saturated C16-18 fatty alcohols with an average degree of ethoxylation of 12 to 30, characterized in that the weight ratio of the emulsifier a) to the emulsifier b) is 2:1 to 1:5, preferably 1:1 to 1:2.

Use of a cosmetic composition including a) at least one emulsifier a) from the group of ethoxylated unsaturated C16-18 fatty alcohols with an average degree of ethoxylation of 4 to 6; b) at least one emulsifier from the group of saturated C16-18 fatty alcohols with an average degree of ethoxylation of 12 to 30, for temporary shaping of keratin-containing fibers, in particular human hair.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject matter of the present patent application is a cosmetic composition for the temporary shaping of keratin-containing fibers, including:
a) at least one emulsifier a) from the group of ethoxylated unsaturated C16-18 fatty alcohols with an average degree of ethoxylation of 4 to 6;
b) at least one emulsifier b) from the group of ethoxylated saturated C16-18 fatty alcohols with an average degree of ethoxylation of 12 to 30, characterized in that the weight ratio of the emulsifier a) to the emulsifier b) is 2:1 to 1:5, preferably 1:1 to 1:2.

The “average degree of ethoxylation” refers to the molar amount of ethylene oxide reacted with one mole fatty alcohol. The degree of ethoxylation is thus a statistical mean which may correspond to an integer or a fractional number for a specific product.

A first essential component of cosmetic compositions according to the invention is the emulsifier a) from the group of ethoxylated unsaturated C16-18 fatty alcohols with an average degree of ethoxylation of 4 to 6. The use of an ethoxylated oleyl alcohol as emulsifier a) is preferred. Especially preferred are in particular the ethoxylated fatty alcohols with the INCI designations oleth-4, oleth-5 and oleth-6. In preferred cosmetic compositions the emulsifier a) has an average degree of ethoxylation of 5. A most especially preferred emulsifier a) is the ethoxylated fatty alcohol with the INCI designation oleth-5.

Cosmetic, optical and haptic properties are influenced advantageously by the amount by weight of emulsifier a) in the total weight of the cosmetic composition. Especially preferred cosmetic compositions according to the invention are characterized in that they contain, based on their total weight, 4.0 to 16% by weight, preferably 6.0 to 14% by weight and in particular 8.0 to 12% by weight of emulsifier a).

As the second essential ingredient, the cosmetic compositions according to the invention contain an emulsifier b) from the group of ethoxylated saturated C16-18 fatty alcohols with an average degree of ethoxylation of 12 to 30. Preferred fatty alcohols are selected from the group of cetyl alcohol (hexadecanol) and stearyl alcohol (octadecanol). The C16-18 fatty alcohols may be used either alone or in mixture. In particular, the ethoxylated fatty alcohols with the INCI designations ceteth-15, ceteth-16, ceteth-20, isoceteth-20, ceteareth-12, ceteareth-15, ceteareth-20, ceteareth-23, ceteareth-25, ceteareth-30, steareth-16, steareth-20, steareth-21, steareth-25 and isosteareth-20 are preferred, wherein the ethoxylated fatty alcohols with the INCI designations ceteth-20, isoceteth-20, ceteareth-20, steareth-20 and isosteareth-20 are especially preferred. Cosmetic composition according to any one of the preceding claims, characterized in that the emulsifier b) has an average degree of ethoxylation of 15 to 25, preferably 20. A most especially preferred emulsifier b) is the ethoxylated fatty alcohol with the INCI designation ceteth-20.

The amount by weight of emulsifier b) in the total weight of the cosmetic compositions according to the invention is preferably 6.0 to 20% by weight, preferably 8.0 to 18% by weight and in particular 10 to 16% by weight. These amounts by weight have proven to be advantageous for the cosmetic, optical and haptic properties of the compositions according to the invention.

Preferred cosmetic compositions contain as an additional ingredient at least one fat c). Preferred cosmetic compositions are characterized by including a fat c) from the group of mineral, natural and synthetic oils, in particular especially preferably from the group of mineral oils.

Triglycerides and mixtures of triglycerides may be used as natural (vegetable) oils. Preferred natural oils include coconut oil, (sweet) almond oil, walnut oil, peach kernel oil, apricot kernel oil, avocado oil, tree tea oil, soy oil, sesame oil, sunflower oil, tsubaki oil, evening primrose oil, rice bran oil, palm kernel oil, mango kernel oil, cardamine pratensis oil, thistle oil, macadamia nut oil, grape seed oil, amaranth seed oil, argan oil, bamboo oil, olive oil, wheat germ oil, pumpkin seed oil, mallow oil, hazel nut oil, safflower oil, canola oil, sasanqua oil, jojoba oil, rambutan oil, cocoa butter and shea butter.

Silicone compounds may be considered as the synthetic oils. Suitable silicones can be selected from:
(i) polyalkylsiloxanes, polyarylsiloxanes, polyalkylarylsiloxanes that are volatile or nonvolatile, linear, branched or cyclic, crosslinked or non-crosslinked;
(ii) polysiloxanes having one or more organofunctional groups in their general structure, selected from the following:
   a) substituted or unsubstituted aminated groups;
   b) (per)fluorinated groups;
   c) thiol groups;
   d) carboxylate groups
   e) hydroxylated groups;
   f) alkoxylated groups;
   g) acyloxyalkyl groups;
   h) amphoteric groups;
   i) bisulfate groups;
   j) hydroxyacylamino groups;
   k) carboxyl groups;
   l) sulfonic acid groups and
   m) sulfate or thiosulfate groups;
(iii) linear polysiloxane (A)-polyoxyalkylene (B) block copolymers of the (A-B)ₘ type where n>3;
(iv) grafted silicone polymers with an organic backbone structure that does not contain silicone and consists of an organic main chain formed from organic monomers which do not contain any silicone and onto which at least one polysiloxane macromer has been grafted in the chain and optionally also on at least one chain terminus;
(v) grafted silicone polymers with a basic polysiloxane structure, onto which organic monomers that do not contain silicone and have a polysiloxane main chain onto which at least one organic monomer has been grafted in the chain and optionally onto at least one of its termini;
(vi) or mixtures thereof.

The mineral oils used may in particular comprise mineral oils, paraffin oils and isoparaffin oils as well synthetic hydrocarbons. Especially preferred are paraffins, in particular liquid paraffins, preferably paraffins with the INCI designation paraffinum liquidum. The amount by weight of the fat c) in the total weight of the cosmetic compositions according to the invention is preferably 6.0 to 22% by weight, preferably 8.0 to 20% by weight and in particular 10 to 28% by weight. Especially preferred cosmetic compositions are characterized in that they contain, based on their total weight, 6.0 to 22% by weight, preferably 8.0 to 20% by weight and in particular 10 to 28% by weight of a fat c) from the group of mineral, natural and synthetic oils, in particular preferably from the group of mineral oils.

The cosmetic compositions according to the invention are preferably based on an aqueous alcoholic or aqueous-alcoholic vehicle. Cosmetic compositions including, based on their total weight, 20 to 70% by weight, preferably 30 to 60% by weight and in particular 40 to 55% by weight water are preferred. Especially preferred are aqueous or aqueous-alcoholic vehicles. Cosmetic compositions based on aqueous-alcoholic vehicles contain, based on their total weight, 20 to 70% by weight, preferably 30 to 60% by weight and in particular 40 to 55% by weight water and at least one alcohol preferably an alcohol from the group of ethanol, glycerol, 1,3-butylene glycol and sorbitol. Use of polyhydric alcohols from the group of glycerol, 1,3-butylene glycol and sorbitol is preferred. Regardless of their water content, such cosmetic compositions that contain, based on their total weight, 8.0 to 24% by weight, preferably 10 to 22% by weight, and in particular 12 to 20% by weight of at least one polyhydric alcohol d), preferably at least one alcohol from the group of glycerol, 1,3-butylene glycol and sorbitol, are preferred.

The cosmetic compositions according to the invention may contain additional additives, auxiliary substances and care substances. The amount by weight of the additional ingredients contained in the compositions according to the invention in addition to components a) through d), in particular the additional additives, auxiliary substances and care substances in the total weight of the compositions according to the invention contained in these compositions is preferably less than 10% by weight, preferably less than 7.0% by weight, especially preferably less than 5.0% by weight and in particular less than 3.0% by weight. The amount by weight of these additives, auxiliary substances and care substances in the total weight of the cosmetic agent according to the invention may be, for example, 0.1 to 5.0% by weight in particular 0.2 to 3.0% by weight.

The compositions according to the invention may contain one or more film-forming polymers. Film-forming polymers are understood to be polymers which leave a continuous film on the hair after drying. Film-forming polymers are also understood to be those polymers which, when applied in 0.01 to 20% by weight aqueous alcoholic or aqueous-alcoholic solution, are capable of depositing a transparent polymer film on the hair.

Suitable film-forming and/or setting polymers both permanently and temporarily include cationic, anionic, nonionic or amphoteric polymers. The film-forming polymers may be of synthetic or natural origin.

Suitable synthetic film-forming hair setting polymers that are preferred for use according to the invention include homopolymers or copolymers constructed from at least one of the following monomers: vinyl pyrrolidone, vinyl caprolactam, vinyl esters such as vinyl acetate, vinyl alcohol, acrylamide, methacrylamide, C₁ to C₇ alkyl acrylamide, C₁ to C₇ dialkyl acrylamide, C₁ to C₇ alkyl methacrylamide, C₁ to C₇ dialkyl methacrylamide, C₁ to C₇ alkyl acrylate, acrylic acid, propylene glycol and ethylene glycol, wherein the C₁ to C₇ alkyl groups of these monomers are preferably C₁ to C₃ alkyl groups. Examples include the homopolymers of vinyl caprolactam, vinyl pyrrolidone or N-vinyl formamide. Other suitable synthetic film-forming hair-setting polymers include, for example, copolymers of vinyl pyrrolidone and vinyl acetate, vinyl pyrrolidone and styrene or terpolymers of vinyl pyrrolidone, vinyl acetate and vinyl propionate, polyacrylamides. Suitable natural film-forming polymers include, for example, cellulose derivatives, for example, hydroxypropyl cellulose with a molecular weight of 30,000 to 50,000 g/mol.

Additional examples of conventional film-forming polymers include acrylamide/ammonium acrylate copolymer, acrylamides/DMAPA acrylates/methoxy PEG methacrylate copolymer, acrylamidopropyl trimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butyl acrylamide copolymer, acrylates copolymer, acrylates/C₁₋₂ succinates/hydroxyacrylates copolymer, acrylates/lauryl acrylate/stearyl acrylate/ethylamine oxide methacrylate copolymer, acrylates/octylacrylamide copolymer, acrylates/octylacrylamide/diphenyl amodimethicone copolymer, acrylates/stearyl acrylate/ethylamine oxide methacrylate copolymer, acrylates/VA copolymer, acrylates/VP copolymer, adipic acid/diethylene triamine copolymer, adipic acid/dimethylaminohydroxypropyl diethylene triamine copolymer, adipic acid/epoxypropyl diethylene triamine copolymer, adipic acid/isophthalic acid/neopentyl glycol/trimethylolpropane copolymer, allyl stearate/VA copolymer, aminoethyl acrylate phosphate/acrylates copolymer, aminoethyl propanediol acrylates/acrylamide copolymer, aminoethyl propanediol-AMPD acrylates/diacetone acrylamide copolymer, ammonium VA/acrylates copolymer, AMPD-acrylates/diacetone acrylamide copolymer, AMP-acrylates/allyl methacrylate copolymer, AMP-acrylates/C₁₋₁₈ alkyl acrylates/C₁₋₈ alkyl acrylamide copolymer, AMP-acrylates/diacetone acrylamide copolymer, AMP-acrylates/dimethylaminoethyl methacrylate copolymer, bacillus/rice bran extract/soybean extract ferment filtrate, bis-butyloxyamodimethicone/PEG-60 copolymer, butyl acrylate/ethylhexyl methacrylate copolymer, butyl acrylate/hydroxypropyl dimethicone acrylate copolymer, butylated PVP, butyl ester of ethylene/MA copolymer, butyl ester of PVM/MA copolymer, calcium/sodium PVM/MA copolymer, cornstarch/acrylamide/sodium acrylate copolymer, diethylene glycolamine/epichlorohydrin/piperazine copolymer, dimethicone cross polymer, diphenyl amodimethicone, ethyl ester of PVM/MA copolymer, hydrolyzed wheat protein/PVP cross polymer, isobutylene/ethyl maleimide/hydroxyethyl maleimide copolymer, isobutylene/MA copolymer, isobutyl methacrylate/bis-hydroxypropyl dimethicone acrylate copolymer, isopropyl ester of PVM/MA copolymer, lauryl acrylate cross polymer, lauryl methacrylate/glycol dimethacrylate cross polymer, MEA sulfite, methacrylic acid/sodium acryl amidomethyl propane sulfonate copolymer, methacryloyl ethylbetaine/acrylates copolymer, octylacrylamide/acrylates/butylaminoethyl methacrylate copolymer, PEG/PPG-25/25 dimethicone/acrylates copolymer, PEG-8/SMDI copolymer, polyacrylamide, polyacrylate-6, poly-f3-alanine/glutaric acid cross-polymer, polybutylene terephthalate, polyester-1, polymethyl acrylate, polyethylene terephthalate, polymethacryloyl ethylbetaine, polypentaerythrityl terephthalate, polyperfluoroperhydrophenanthrene, polyquatemium-1, polyquatemium-2, polyquaternium-4, polyquatemium-5, polyquaternium-6, polyquaternium-7, polyquatemium-8, polyquaternium-9, polyquatemium-10, polyquatemium-11, polyquatemium-12, polyquatemium-13, polyquatemium-14, polyquatemium-15, polyquaternium-16, polyquatemium-17, polyquatemium-18, polyquatemium-19, polyquaternium-20, polyquaternium-22, polyquaternium-24, polyquaternium-27, polyquaternium-28, polyquatemium-29, polyquatemium-30, polyquaternium-31, polyquatemium-32, polyquatemium-33, polyquatemium-34, polyquatemium-35, polyquatemium-36, polyquatemium-37, polyquatemium-39, polyquaternium-45, polyquaternium-46, polyquatemium-47, polyquaternium-48, polyquaternium-49, polyquatemium-50, polyquatemium-55, polyquatemium-56, polysilicone-9, polyurethane-1, polyurethane-6, polyurethane-10, polyvinyl acetate, polyvinyl butyral, polyvinyl caprolactam, polyvinyl formamide, polyvinyl imidazolinium acetate, polyvinyl methyl ether, potassium butyl ester of PVM/MA copolymer, potassium ethyl ester of PVM/MA copolymer, PPG-70 polyglyceryl-10 ether, PPG-12/SMDI copolymer, PPG-51/SMDI copolymer, PPG-10 sorbitol, PVM/MA copolymer, PVP, PVPNA/itaconic acid copolymer, PVPNA/vinyl propionate copolymer, rhizobian gum, rosin acrylate, shellac, sodium butyl ester of PVM/MA copolymer, sodium ethyl ester of PVM/MA copolymer, sodium polyacrylate, sterculia urens gum, terephthalic acid/isophthalic acid/sodium isophthalic acid sulfonate/glycol copolymer, trimethylol propane triacrylate, trimethylsiloxysilyl carbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinyl amine/vinyl alcohol copolymer, vinyl caprolactam/VP/dimethylaminoethyl methacrylate copolymer, VP/acrylates/lauryl methacrylate copolymer, VP/dimethylaminoethyl methacrylate copolymer, VP/DMAPA acrylates copolymer, VP/hexadecene copolymer, VP/VA copolymer, VP/vinyl caprolactam/DMAPA acrylates copolymer, yeast palmitate and styrene/VP copolymer.

The compositions according to the invention in particular preferably additionally contain at least one styrene/VP copolymer and/or one vinyl pyrrolidone-vinyl acetate copolymer, preferably a styrene/VP copolymer.

The compositions according to the invention contain the film-forming polymer preferably in amount of less than 5.0% by weight, preferably in amounts of less than 3.0% by weight, most especially preferably in amounts between 0.01 and 2.0% by weight and in particular in amounts between 0.04 and 1.0% by weight, each based on their total weight.

Preferred compositions according to the invention are characterized in that they contain at least one organosiloxane-oxyalkylene copolymer. Such organosiloxane-oxyalkylene copolymers are available, for example, under the brand name Dow Corning 193 (INCI: PEG-12 dimethicone) or Abil B 8843 (INCI: PEG-14 dimethicone).

The amount by weight of the organosiloxane-oxyalkylene copolymer in the total weight of the compositions according to the invention is preferably 0.01 to 2.0% by weight, preferably 0.02 to 1.0% by weight and in particular 0.05 to 0.5% by weight.

As an additional care substance, the composition may contain a protein hydrolysate and/or one of its derivatives. Protein hydrolysates are product mixtures obtained by acetic, basic or enzymatically catalyzed degradation of proteins (albumins). The term “protein hydrolysate” is understood according to the invention to also include total hydrolysates as well as individual amino acids and their derivatives as well as mixtures of various amino acids. The molecular weight of the protein hydrolysates that can be used according to the invention is between 75, the molecular weight of glycine and 200,000. The molecular weight is preferably 75 to 50,000 and most especially preferably 75 to 20,000 Dalton.

The composition according to the invention may also contain at least one vitamin, a provitamin, a vitamin precursor and/or one of their derivatives as a care substance. According to the invention, vitamins, provitamins and vitamin precursors, which are usually assigned to groups A, B, C, E, F and H, are preferred.

The compositions according to the invention may also contain at least one plant extract but also monosaccharides and/or oligosaccharides and/or lipids as the care substance.

The compositions according to the invention may contain as a care substance at least one UV filter. The UV filters are usually contained in the compositions according to the invention in amounts of 0.01-1% by weight, based on the powdered composition according to the invention. Amounts of 0.1-0.5% by weight are preferred.

A few preferred cosmetic compositions according to the invention with a weight ratio of emulsifier a) to emulsifier b) between 2:1 and 1:5, preferably 1:1 to 1:2 can be taken from the following tables (amounts in % by weight based on the total weight of the cleaning agent, unless otherwise indicated).

| | Formula | Formula | Formula | Formula | Formula |
|---|---|---|---|---|---|
| | 1 | 2 | 3 | 4 | 5 |
| Emulsifier a)* | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
**Emulsifier b) from the group of ethoxylated saturated C16-18 fatty alcohols with an average degree of ethoxylation of 12 to 30

| | Formula | Formula | Formula | Formula | Formula |
|---|---|---|---|---|---|
| | 6 | 7 | 8 | 9 | 10 |
| Oleth-5 | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Miscellaneous | add 100 | add 100 | add 100 | add 100 | add 100 |

**Emulsifier b) from the group of ethoxylated saturated C16-18 fatty alcohols with an average degree of ethoxylation of 12 to 30

| | Formula | Formula | Formula | Formula | Formula |
|---|---|---|---|---|---|
| | 11 | 12 | 13 | 14 | 15 |
| Emulsifier a)* | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6

| | Formula | Formula | Formula | Formula | Formula |
|---|---|---|---|---|---|
| | 16 | 17 | 18 | 19 | 20 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Miscellaneous | add 100 | add 100 | add 100 | add 100 | add 100 |

| | Formula | Formula | Formula | Formula | Formula |
|---|---|---|---|---|---|
| | 21 | 22 | 23 | 24 | 25 |
| Emulsifier a)* | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
**Emulsifier b) from the group of ethoxylated saturated C16-18 fatty alcohols with an average degree of ethoxylation of 12 to 30
***Fat c) from the group of mineral, natural and synthetic oils

| | Formula | Formula | Formula | Formula | Formula |
|---|---|---|---|---|---|
| | 26 | 27 | 28 | 29 | 30 |
| Oleth-5 | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Miscellaneous | add 100 | add 100 | add 100 | add 100 | add 100 |

**Emulsifier b) from the group of ethoxylated saturated C16-18 fatty alcohols with an average degree of ethoxylation of 12 to 30
***Fat c) from the group of mineral, natural and synthetic oils

| | Formula | Formula | Formula | Formula | Formula |
|---|---|---|---|---|---|
| | 31 | 32 | 33 | 34 | 35 |
| Emulsifier a)* | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
***Fat c) from the group of mineral, natural and synthetic oils

| | Formula | Formula | Formula | Formula | Formula |
|---|---|---|---|---|---|
| | 36 | 37 | 38 | 39 | 40 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Miscellaneous | add 100 | add 100 | add 100 | add 100 | add 100 |

***Fat c) from the group of mineral, natural and synthetic oils

| | Formula | Formula | Formula | Formula | Formula |
|---|---|---|---|---|---|
| | 41 | 42 | 43 | 44 | 45 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Paraffinum | 6.0 to 22 | 8.0 to 20 | 10 to 28 | 12 | 14 |
| Liquidum | | | | | |
| Miscellaneous | add 100 | add 100 | add 100 | add 100 | add 100 |

| | Formula | Formula | Formula | Formula | Formula |
|---|---|---|---|---|---|
| | 46 | 47 | 48 | 49 | 50 |
| Emulsifier a)* | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Water | 20 to 70 | 30 to 60 | 40 to 55 | 45 | 43 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
**Emulsifier b) from the group of ethoxylated saturated C16-18 fatty alcohols with an average degree of ethoxylation of 12 to 30
***Fat c) from the group of mineral, natural and synthetic oils

| | Formula | Formula | Formula | Formula | Formula |
|---|---|---|---|---|---|
| | 51 | 52 | 53 | 54 | 55 |
| Oleth-5 | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Water | 20 to 70 | 30 to 60 | 40 to 55 | 45 | 43 |
| Miscellaneous | add 100 | add 100 | add 100 | add 100 | add 100 |

**Emulsifier b) from the group of ethoxylated saturated C16-18 fatty alcohols with an average degree of ethoxylation of 12 to 30
***Fat c) from the group of mineral, natural and synthetic oils

| | Formula | Formula | Formula | Formula | Formula |
|---|---|---|---|---|---|
| | 56 | 57 | 58 | 59 | 60 |
| Emulsifier a)* | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Water | 20 to 70 | 30 to 60 | 40 to 55 | 45 | 43 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
***Fat c) from the group of mineral, natural and synthetic oils

| | Formula | Formula | Formula | Formula | Formula |
|---|---|---|---|---|---|
| | 61 | 62 | 63 | 64 | 65 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Water | 20 to 70 | 30 to 60 | 40 to 55 | 45 | 43 |
| Miscellaneous | add 100 | add 100 | add 100 | add 100 | add 100 |

***Fat c) from the group of mineral, natural and synthetic oils

| | Formula | Formula | Formula | Formula | Formula |
|---|---|---|---|---|---|
| | 66 | 67 | 68 | 69 | 70 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Paraffinum | 6.0 to 22 | 8.0 to 20 | 10 to 28 | 12 | 14 |
| liquidum | | | | | |
| Water | 20 to 70 | 30 to 60 | 40 to 55 | 45 | 43 |
| Miscellaneous | add 100 | add 100 | add 100 | add 100 | add 100 |

| | Formula | Formula | Formula | Formula | Formula |
|---|---|---|---|---|---|
| | 71 | 72 | 73 | 74 | 75 |
| Emulsifier a)* | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Alcohol d)**** | 8.0 to 24 | 10 to 22 | 12 to 20 | 16 | 12 |
| Water | 20 to 70 | 30 to 60 | 40 to 55 | 45 | 43 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
**Emulsifier b) from the group of ethoxylated saturated C16-18 fatty alcohols with an average degree of ethoxylation of 12 to 30
***Fat c) from the group of mineral, natural and synthetic oils
****Alcohol from the group of glycerol, 1,3-butylene glycol and sorbitol

| | Formula | Formula | Formula | Formula | Formula |
|---|---|---|---|---|---|
| | 76 | 77 | 78 | 79 | 80 |
| Oleth-5 | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Alcohol d)**** | 8.0 to 24 | 10 to 22 | 12 to 20 | 16 | 12 |
| Water | 20 to 70 | 30 to 60 | 40 to 55 | 45 | 43 |
| Miscellaneous | add 100 | add 100 | add 100 | add 100 | add 100 |

**Emulsifier b) from the group of ethoxylated saturated C16-18 fatty alcohols with an average degree of ethoxylation of 12 to 30
***Fat c) from the group of mineral, natural and synthetic oils
****Alcohol from the group of glycerol, 1,3-butylene glycol and sorbitol

| | Formula | Formula | Formula | Formula | Formula |
|---|---|---|---|---|---|
| | 81 | 82 | 83 | 84 | 85 |
| Emulsifier a)* | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Alcohol d)**** | 8.0 to 24 | 10 to 22 | 12 to 20 | 16 | 12 |
| Water | 20 to 70 | 30 to 60 | 40 to 55 | 45 | 43 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
***Fat c) from the group of mineral, natural and synthetic oils
****Alcohol from the group of glycerol, 1,3-butylene glycol and sorbitol

| | Formula | Formula | Formula | Formula | Formula |
|---|---|---|---|---|---|
| | 86 | 87 | 88 | 89 | 90 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Alcohol d)**** | 8.0 to 24 | 10 to 22 | 12 to 20 | 16 | 12 |
| Water | 20 to 70 | 30 to 60 | 40 to 55 | 45 | 43 |
| Miscellaneous | add 100 | add 100 | add 100 | add 100 | add 100 |

***Fat c) from the group of mineral, natural and synthetic oils
****Alcohol from the group of glycerol, 1,3-butylene glycol and sorbitol

| | Formula | Formula | Formula | Formula | Formula |
|---|---|---|---|---|---|
| | 91 | 92 | 93 | 94 | 95 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Paraffinum | 6.0 to 22 | 8.0 to 20 | 10 to 28 | 12 | 14 |
| liquidum | | | | | |
| Alcohol d)**** | 8.0 to 24 | 10 to 22 | 12 to 20 | 16 | 12 |
| Water | 20 to 70 | 30 to 60 | 40 to 55 | 45 | 43 |
| Miscellaneous | add 100 | add 100 | add 100 | add 100 | add 100 |

****Alcohol from the group of glycerol, 1,3-butylene glycol and sorbitol

| | Formula | Formula | Formula | Formula | Formula |
|---|---|---|---|---|---|
| | 96 | 97 | 98 | 99 | 100 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Glycerol | 0.5 to 6.0 | 1.0 to 5.0 | 1.0 to 3.0 | 3.0 | 2.0 |
| Water | 20 to 70 | 30 to 60 | 40 to 55 | 45 | 43 |
| Miscellaneous | add 100 | add 100 | add 100 | add 100 | add 100 |

***Fat c) from the group of mineral, natural and synthetic oils

| | Formula | Formula | Formula | Formula | Formula |
|---|---|---|---|---|---|
| | 101 | 102 | 103 | 104 | 105 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Sorbitol | 1.0 to 20 | 2.0 to 15 | 5.0 to 12 | 9.0 | 9.0 |
| Water | 20 to 70 | 30 to 60 | 40 to 55 | 45 | 43 |
| Miscellaneous | add 100 | add 100 | add 100 | add 100 | add 100 |

***Fat c) from the group of mineral, natural and synthetic oils

| | Formula | Formula | Formula | Formula | Formula |
|---|---|---|---|---|---|
| | 106 | 107 | 108 | 109 | 110 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| 1,3-Butylene | 0.5 to 20 | 1.0 to 12 | 2.0 to 8.0 | 5.0 | 5.0 |
| glycol | | | | | |
| Water | 20 to 70 | 30 to 60 | 40 to 55 | 45 | 43 |
| Miscellaneous | add 100 | add 100 | add 100 | add 100 | add 100 |

***Fat c) from the group of mineral, natural and synthetic oils

| | Formula | Formula | Formula | Formula | Formula |
|---|---|---|---|---|---|
| | 111 | 112 | 113 | 114 | 115 |
| Emulsifier a)* | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Glycerol | 0.5 to 6.0 | 1.0 to 5.0 | 1.0 to 3.0 | 3.0 | 2.0 |
| Sorbitol | 1.0 to 20 | 2.0 to 15 | 5.0 to 12 | 9.0 | 9.0 |
| 1,3-Butylene | 0.5 to 20 | 1.0 to 12 | 2.0 to 8.0 | 5.0 | 5.0 |
| glycol | | | | | |
| Water | 20 to 70 | 30 to 60 | 40 to 55 | 45 | 43 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
**Emulsifier b) from the group of ethoxylated saturated C16-18 fatty alcohols with an average degree of ethoxylation of 12 to 30
***Fat c) from the group of mineral, natural and synthetic oils

| | Formula | Formula | Formula | Formula | Formula |
|---|---|---|---|---|---|
| | 116 | 117 | 118 | 119 | 120 |
| Oleth-5 | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Glycerol | 0.5 to 6.0 | 1.0 to 5.0 | 1.0 to 3.0 | 3.0 | 2.0 |
| Sorbitol | 1.0 to 20 | 2.0 to 15 | 5.0 to 12 | 9.0 | 9.0 |
| 1,3-Butylene | 0.5 to 20 | 1.0 to 12 | 2.0 to 8.0 | 5.0 | 5.0 |
| glycol | | | | | |
| Water | 20 to 70 | 30 to 60 | 40 to 55 | 45 | 43 |
| Miscellaneous | add 100 | add 100 | add 100 | add 100 | add 100 |

**Emulsifier b) from the group of ethoxylated saturated C16-18 fatty alcohols with an average degree of ethoxylation of 12 to 30
***Fat c) from the group of mineral, natural and synthetic oils

| | Formula | Formula | Formula | Formula | Formula |
|---|---|---|---|---|---|
| | 121 | 122 | 123 | 124 | 125 |
| Emulsifier a)* | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Fat c)*** | 6.0 to 22 | 8.0 to 20 | 10 to 28 | 12 | 14 |
| Glycerol | 0.5 to 6.0 | 1.0 to 5.0 | 1.0 to 3.0 | 3.0 | 2.0 |
| Sorbitol | 1.0 to 20 | 2.0 to 15 | 5.0 to 12 | 9.0 | 9.0 |
| 1,3-Butylene | 0.5 to 20 | 1.0 to 12 | 2.0 to 8.0 | 5.0 | 5.0 |
| glycol | | | | | |
| Water | 20 to 70 | 30 to 60 | 40 to 55 | 45 | 43 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
***Fat c) from the group of mineral, natural and synthetic oils

| | Formula | Formula | Formula | Formula | Formula |
|---|---|---|---|---|---|
| | 126 | 127 | 128 | 129 | 130 |
| Emulsifier a)* | 4.0 to 16 | 6.0 to 14 | 8.0 to 12 | 12 | 10 |
| Emulsifier b)** | 6.0 to 20 | 8.0 to 18 | 10 to 16 | 8.0 | 12.5 |
| Paraffinum | 6.0 to 22 | 8.0 to 20 | 10 to 28 | 12 | 14 |
| liquidum | | | | | |
| Glycerol | 0.5 to 6.0 | 1.0 to 5.0 | 1.0 to 3.0 | 3.0 | 2.0 |
| Sorbitol | 1.0 to 20 | 2.0 to 15 | 5.0 to 12 | 9.0 | 9.0 |
| 1,3-Butylene | 0.5 to 20 | 1.0 to 12 | 2.0 to 8.0 | 5.0 | 5.0 |
| glycol | | | | | |
| Water | 20 to 70 | 30 to 60 | 40 to 55 | 45 | 43 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6
**Emulsifier b) from the group of ethoxylated saturated C16-18 fatty alcohols with an average degree of ethoxylation of 12 to 30

| | Formula | Formula | Formula | Formula | Formula |
|---|---|---|---|---|---|
| | 131 | 132 | 133 | 134 | 135 |
| Emulsifier a)* | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Paraffinum | 6.0 to 22 | 8.0 to 20 | 10 to 28 | 12 | 14 |
| liquidum | | | | | |
| Glycerol | 0.5 to 6.0 | 1.0 to 5.0 | 1.0 to 3.0 | 3.0 | 2.0 |
| Sorbitol | 1.0 to 20 | 2.0 to 15 | 5.0 to 12 | 9.0 | 9.0 |
| 1,3-Butylene | 0.5 to 20 | 1.0 to 12 | 2.0 to 8.0 | 5.0 | 5.0 |
| glycol | | | | | |
| Water | 20 to 70 | 30 to 60 | 40 to 55 | 45 | 43 |
| Miscellaneous | add 100 | add 100 | add 100 | add 100 | add 100 |

*Emulsifier a) from the group of ethoxylated unsaturated C16-18 fatty alcohols with an average degree of ethoxylation of 4 to 6

| | Formula | Formula | Formula | Formula | Formula |
|---|---|---|---|---|---|
| | 136 | 137 | 138 | 139 | 140 |
| Oleth-5 | 4.0 to 16 | 6.0 to 14 | 8.0 to 12 | 12 | 10 |
| Ceteth-20 | 6.0 to 20 | 8.0 to 18 | 10 to 16 | 8.0 | 12.5 |
| Paraffinum | 6.0 to 22 | 8.0 to 20 | 10 to 28 | 12 | 14 |
| liquidum | | | | | |
| Glycerol | 0.5 to 6.0 | 1.0 to 5.0 | 1.0 to 3.0 | 3.0 | 2.0 |
| Sorbitol | 1.0 to 20 | 2.0 to 15 | 5.0 to 12 | 9.0 | 9.0 |
| 1,3-Butylene | 0.5 to 20 | 1.0 to 12 | 2.0 to 8.0 | 5.0 | 5.0 |
| glycol | | | | | |
| Water | 20 to 70 | 30 to 60 | 40 to 55 | 45 | 43 |
| Miscellaneous | add 100 | add 100 | add 100 | add 100 | add 100 |

Especially preferred cosmetic compositions according to the invention are in the form of a microemulsion.

Preferred cosmetic preparations according to the invention are transparent. Preparations having a turbidity of ≦250 NTU (nephelometric turbidity unit), preferably a turbidity of ≦150 NTU, especially preferably a turbidity of ≦100 NTU, in particular a turbidity of ≦50 NTU and most especially preferably ≦20 NTU are referred to as being “transparent.” The turbidity value can be determined using a 2100P turbidimeter from the company Hach, for example.

Especially preferred cosmetic compositions according to the invention are in the form of microemulsions having a turbidity of ≦250 NTU (nephelometric turbidity unit), preferably a turbidity of ≦150 NTU, especially preferably a turbidity of ≦100 NTU, in particular a turbidity of ≦50 NTU and most especially preferably ≦20 NTU. The turbidity value can be determined, for example, using a 2100P turbidimeter from the company Hach.

Preferred cosmetic preparations according to the invention have a hardness between 0.7 and 1.1 mm, preferably between 0.8 and 1.0 mm, measured with a texture analyzer. Cosmetic preparations with a corresponding hardness can be metered easily, for example, by removal from a crucible using one's fingers, and they permit a remodelable shaping of the keratinic fibers.

The hardness of the cosmetic preparation is measured by means of the texture analyzer at 21° C. and 50% relative atmospheric humidity in a specimen volume of 75 ml and a specimen height of 3 cm. The cylindrical test body (7 mm diameter) is advanced at a test rate of 1 mm/sec into the specimen. The force limit value at which the data acquisition begins is 3 g. The forward advance stops at a force limit value of 23 g. The hardness of the cosmetic preparation is obtained as the difference between the depth of penetration of the sample body into the preparation at a force of 23 g minus the depth of penetration of the specimen body into the preparation at a force of 3 g.

As explained above, the cosmetic preparations according to the invention are suitable in particular for preshaping keratinic fibers. Another subject matter of this patent application is therefore the use of a cosmetic composition including:
a) at least one emulsifier a) from the group of ethoxylated unsaturated C16-18 fatty alcohols with an average degree of ethoxylation of 4 to 6;
b) at least one emulsifier from the group of saturated C16-18 fatty alcohols with an average degree of ethoxylation of 12 to 30,
   for temporary shaping of keratin-containing fibers, in particular human hair.

Temporary shaping of the keratinic fibers is associated with an improvement in the feel and sheen of the fibers. The improved feel and sheen can be detected not only in comparison with untreated hair but also in comparison with cosmetic compositions of the state of the art. Another subject matter of this patent application is therefore the use of a cosmetic composition including
a) at least one emulsifier a) from the group of ethoxylated unsaturated C16-18 fatty alcohols with an average degree of ethoxylation of 4 to 6;
b) at least one emulsifier from the group of saturated C16-18 fatty alcohols with an average degree of ethoxylation of 12 to 30,
   to improve the feel and/or sheen of keratin-containing fibers, in particular human hair.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for the temporary shaping of keratin-containing fibers, comprising:
a) at least one emulsifier a) selected from the group consisting of ethoxylated unsaturated C16-C18 fatty alcohols with an average degree of ethoxylation of 4 to 6;
b) at least one emulsifier b) selected from the group consisting of ethoxylated saturated C16-18 fatty alcohols with an average degree of ethoxylation of 12 to 30,
wherein the weight ratio of the emulsifier a) to the emulsifier b) is 2:1 to 1:5, preferably 1:1 to 1:2.

2. The cosmetic composition according to claim 1, wherein the emulsifier a) has an average degree of ethoxylation of 5.

3. The cosmetic composition according to claim 1, wherein the composition comprises, based on its total weight, 4.0 to 16% by weight of the emulsifier a).

4. The cosmetic composition according to claim 1, wherein the composition comprises, based on its total weight, 6.0 to 14% of emulsifier a).

5. The cosmetic composition according to claim 1, wherein the composition comprises, based on its total weight, 8.0 to 12% of emulsifier a).

6. The cosmetic composition according to claim 1, wherein the emulsifier b) has an average degree of ethoxylation of 15 to 25.

7. The cosmetic composition according to claim 1, wherein the emulsifier b) has an average degree of ethoxylation of 20.

8. The cosmetic composition according to claim 1, wherein the composition comprises, based on its total weight, 6.0 to 20% by weight of emulsifier b).

9. The cosmetic composition according to claim 1, wherein the composition comprises, based on its total weight, 8.0 to 18% of emulsifier b).

10. The cosmetic composition according to claim 1, wherein the composition comprises, based on its total weight, 10 to 16% of emulsifier b).

11. The cosmetic composition according to claim 1, wherein the composition comprises, based on its total weight, 20 to 70% water.

12. The cosmetic composition according to claim 1, wherein the composition comprises, based on its total weight, 30 to 60% water.

13. The cosmetic composition according to claim 1, wherein the composition comprises, based on its total weight, 40 to 55% water.

14. The cosmetic composition according to claim 1, wherein the composition further comprises, based on its total weight, 6.0 to 22% by weight, of a fat c) selected from from the group consisting of mineral oils, natural oils, synthetic oils, and mixtures thereof.

15. The cosmetic composition according to claim 1, wherein the composition further comprises, based on its total weight, 8.0 to 20% by weight, of a fat c) selected from from the group consisting of mineral oils, natural oils, synthetic oils, and mixtures thereof.

16. The cosmetic composition according to claim 1, wherein the composition further comprises, based on its total weight, 8.0 to 24% by weight, of at least one polyhydric alcohol d).

17. The cosmetic composition according to claim 16, wherein the polyhydric alcohol is selected from the group consisting of glycerol, 1,3-butylene glycol, and sorbitol.

18. The cosmetic composition according to claim 1, wherein the cosmetic composition is in the form of a microemulsion.

19. A method for the temporary shaping of keratin-containing fibers, comprising: applying to keratin-containing fibers a cosmetic composition, comprising
a) at least one emulsifier a) from the group of ethoxylated unsaturated C16-18 fatty alcohols with an average degree of ethoxylation of 4 to 6; and
b) at least one emulsifier from the group of saturated C16-18 fatty alcohols with an average degree of ethoxylation of 12 to 30.
